(19) United States
(12) Patent Application Publication
(10) Pub. No.: US 2010/0028985 A1
(43) Pub. Date: Feb. 4, 2010

(54) Reaction Vessel, Reaction Vessel Processing Apparatus and Diagnostic Apparatus

(75) Inventors: Nobuhiro Hanafusa, Kyoto (JP); Koretsugu Ogata, Kyoto (JP); Ryuh Konoshita, Kyoto (JP); Hiroyuki Kuroki, Tokyo (JP); Rika Satou, Tokyo (JP); Ryoko Imagawa, Tokyo (JP); Yusuke Nakamura, Kanagawa (JP); Yozo Ohnishi, Kanagawa (JP)

Correspondence Address: Cheng Law Group, PLLC, 1100 17th Street, N.W., Suite 503, Washington, DC, 20036, US

(73) Assignees: SHIMADZU CORPORATION, KYOTO (JP); TOPPAN PRINTING CO., LTD., TOKYO (JP); RIKEN, Saitama (JP)

(21) Appl. No.: 11/887,387

(22) PCT Filed: Mar. 29, 2006
(86) PCT No.: PCT/JP2006/306550

§ 371 Date: Nov. 7, 2008

(30) Foreign Application Priority Data
Mar. 29, 2005 (JP) 2005-096460

(51) Int. Cl. C12M 1/00

(52) U.S. Cl. 435/287.2

(57) ABSTRACT

A reaction vessel suitable for automating of various measurements is provided. In a preferred embodiment, on the same side of plate-like substrate (10), sample injection part (12), typing reagent reservoir part (14) and mineral oil reservoir part (16) are provided as concaves, and further, multiple probe arrangement parts (18) are provided. The typing reagent reservoir part (14) and mineral oil reservoir part (16) are sealed with film (20). The surface of the substrate (10) is covered with detachable sealing material (22) with a size capable of covering the sample injection part (12), typing reagent reservoir part (14), mineral oil reservoir part (16) and multiple probe arrangement parts (18) in such a manner that the film (20) is covered by the sealing material (22). Liquid transfer is carried out through nozzles.

### TECHNICAL FIELD

The present invention relates to a reaction vessel which is suited for various on-site automatic analyses of, for example, such as chemical reactions, for carrying out genetic analysis research and clinic, and a reaction vessel processing apparatus using the same, for detecting a polymorphism of genome DNA of animals including human beings, and plants, particularly an SNP (single-nucleotide polymorphism), an apparatus for diagnosing disease morbidity and the relationship between the type and effect or side effect of a drug administered by using the detection result of gene polymorphism.

### BACKGROUND ART

A method and apparatus for estimating susceptibility to diseases, etc., by using gene polymorphism have been proposed as follows:

For determining whether a patient is susceptible to sepsis and/or rapidly develops sepsis, a nucleic acid sample is collected from the patient, a pattern 2 allelic gene or a marker gene which is in linkage disequilibrium with a pattern 2 allelic gene in the sample is detected, and if a pattern 2 allelic gene or a marker gene in linkage disequilibrium with a pattern 2 allelic gene is detected, the patient is judged to be susceptible to sepsis (see Patent Literature 1).

For diagnosis of one or more single-nucleotide polymorphisms in the human flt-1 gene, a sequence of one or more positions in human nucleic acid, that is, positions 1953, 3453, 3888 (which are respectively in accordance with numbering in EMBL Accession No. X51602), 519, 786, 1422, 1429 (which are respectively in accordance with numbering in EMBL Accession No. D64016), 454 (in accordance with Sequence No. 3) and 696 (in accordance with Sequence No.: 5) is determined, and by referring to the polymorphism in fl1-1 gene, the constitution of the human is determined (JP-A 2001-299366).

Many methods have, been reported on typing, that is, discrimination of bases in SNP sites. A typical example of these methods is as follows:

For carrying out typing several hundred thousand SNP sites with a relatively small amount of genome DNA, a plurality of base sequences containing at least one single-nucleotide polymorphism are amplified simultaneously with a genome DNA and pairs of primer, and a plurality of base sequences thus amplified are used to discriminate bases in single-nucleotide polymorphic sites contained in the base sequences by a typing step. For the typing step, an invader method or TaqMan PCR is used (see Patent Literature 3).

### Patent Literature 1: Japanese Patent Application National Publication (Laid-Open) No. 2002-533096

### Patent Literature 2: JP-A 2001-299366

### Patent Literature 3: JP-A 2002-300894

### Patent Literature 4: Japanese Patent No. 3452717

Non-Patent Literature 1: Hsu T. M., Law S. M, Duan S, Neri B. P., Kwok P. Y., “Genotyping single-nucleotide polymorphisms by the invader assay with dual-color fluorescence polarization detection”, Clin. Chem., 2001 August; 47(8):1373-7

### DISCLOSURE OF THE INVENTION

### Problems to be Solved by the Invention

The first object of the present invention is to provide a reaction vessel suited for automating measurement of a chemical reaction, and detection of a gene polymorphism.

The second object of the present invention is to provide an apparatus that automates measurement of a chemical reaction, and detection of a gene polymorphism using the reaction vessel of the present invention.

The third object of the present invention is to provide an apparatus that automatically diagnoses disease morbidity, and a relationship between the type and effect or side effect of a drug administered, based on the detection result of a gene polymorphism according to the present invention.

### Means for Solving the Problems

In order to achieve the first object, a reaction vessel of the present invention includes at least one reaction part formed on a plate-like substrate, for allowing a reaction of a sample, and a nonvolatile liquid reservoir part reserving a nonvolatile liquid having a lower specific gravity than a reaction solution, which is formed as a concave portion in the substrate and is sealed with a film.

The reaction vessel of the present invention may further include at least one reagent reservoir part reserving a reagent for use in a reaction of a sample, which is formed as a concave portion in the same substrate and is sealed with a film, to thereby form a reagent kit for reaction of a sample.

In the reaction vessel processing apparatus of the present invention, the reagent or the nonvolatile liquid sealed with a film may be aspirated into a nozzle and transferred to other positions such as a reaction part by insertion of the nozzle through the film or insertion of the nozzle after removal of the film.

The reaction vessel is used for measurement of various reactions including chemical reactions and biochemical reactions. As one application using the present reaction vessel as a reaction reagent kit, detection of a gene polymorphism can be exemplified. The first aspect of the reaction vessel for use in detection of a gene polymorphism is a reaction vessel in which a sample subjected to a gene amplification reaction is injected to the reaction vessel as a biological sample to detect a gene polymorphism. The reaction vessel according to the first aspect includes as a reagent reservoir part, a typing reagent reservoir part reserving a typing reagent prepared in correspondence with a plurality of polymorphic sites, and as a reaction part, a plurality of probe arrangement parts each individually holding a probe emitting fluorescence in correspondence with each of the plurality of polymorphic sites, thereby constituting a gene polymorphism diagnosing reagent kit.

The second aspect of the reaction vessel for use in detection of a gene polymorphism is the reagent kit for reaction of the first aspect further including as a reagent reservoir part, a gene amplification reagent reservoir part for reserving a gene amplification reagent containing a plurality of primers to bind to a plurality of polymorphic sites by sandwiching each site between the primers, and as the reaction part, an amplification reaction part for allowing a gene amplification reaction for a mixture solution of the gene amplification reagent and the sample.

In the gene polymorphism diagnosing reagent kit of the second aspect, a liquid dispensing port of the amplification reaction part preferably has an opening shape that corresponds with the shape of a tip end of the dispensing nozzle, and is made of an elastic material that is capable of closely fitting with the tip end of the dispensing nozzle. Since the amplification reaction part is exposed to repeated cycles of varying temperature, high heat conductivity of the substrate is desired. Accordingly, it is preferred that the thickness of the substrate in the amplification reaction part is smaller than that of the remaining part.

The relationship between the polymorphic sites and primers is as follows: For amplifying one polymorphic site, a pair of primers binding to the polymorphic site by sandwiching it between primers is necessary. A plurality of kinds of polymorphic sites occur in a target biological sample, and when polymorphic sites occur in positions separated from one another, twice as many kinds of primers as kinds of polymorphic sites are necessary. However, when two polymorphic sites are close to each other, amplification thereof can be effected by binding the primers to each of the polymorphic sites by sandwiching each site between the primers or by binding the primers to both sides of a sequence of the two polymorphic sites with no primer between the polymorphic sites. Accordingly, the types of necessary primers are not always twice as many as kinds of polymorphic sites. In the present invention, “a plurality of primers to bind to a plurality of polymorphic sites by sandwiching each site between the primers” is intended to refer to types of primers necessary for amplifying a plurality of polymorphic sites not only in the case where a pair of primers bind to one polymorphic site by sandwiching it between the primers but also in the case where a pair of primers bind to two or more polymorphic sites by sandwiching a series of such polymorphic sites between the primers.

The polymorphism includes mutation, deletion, overlap, trarsfer etc. A typical example is SNP.

Examples of the biological sample include blood, saliva, and genome DNA

One example of the gene amplification reagent is a PCR reagent.

For typing of SNP, adjustment of genome DNA is required at the stage of entering the amplification step, which takes labor and cost. Taking a PCR method for amplifying DNA into account, a direct PCR method which is conducted on a sample such as blood without conducting a pre-treatment is proposed According to this proposal, in a nucleic acid synthesis technique for amplifying an objective gene in a sample containing genes, a gene conjugate in a sample containing genes or a sample containing genes itself is added to a gene amplification reaction solution, and an objective gene in the sample containing genes is amplified at a pH ranging from 8.5 to 9.5 (25° C.) in the reaction solution after addition (see Patent document 4).

In a typing system already constructed, only a small amount of DNA is collected first because a plurality of SNP sites to be typed are amplified by a PCR method; however, it is necessary to carry out a pretreatment for extracting DNA in advance from a biological sample prior to amplification by the PCR method. This takes labor and cost for the pre-treatment.

Such an automated system has not been constructed heretofore that amplifies a plurality of SNP sites to be typed simultaneously when a direct PCR method and a typing method are combined.

The typing step may be achieved by an invader method or a TaqMan PCR method. In such a case, the typing reagent is an invader reagent or a TaqMan PCR reagent.

FIG. 11 schematically shows a detection method for detecting a gene polymorphism using the reaction vessel of the present invention as a gene polymorphism diagnosing reagent kit. In this description, the case where a PCR method is used in an amplification step, and an invader method is used in a typing step. will be explained.

In the PCR step, a PCR regent 4 is added to a biological sample 2 such as blood, or alternatively, the biological sample 2 is added to the PCR reagent 4.

The PCR reagent 4 is prepared in advance, and contains a plurality of primers for SNP sites to be measured, as well as essential reagents such as a pH buffer solution for adjusting pH, four kinds of deoxyribonucleotides, a thermostable synthase, and salts such as MgCl₂ and KCl. Besides the above, substances such as a surfactant and a protein may be added as necessary. The PCR method in the amplification step which may be used in the present invention realizes simultaneous amplification of objective plural SNP sites. The biological sample may or may not be subjected to a nucleic acid extraction procedure. When plural genome DNA containing such SNP sites is amplified by the direct PCR method from a biological sample not subjected to the nucleic acid extraction procedure, a gene amplification reaction regent containing a plurality of primers for such SNP sites is caused to act on the biological sample, and the PCR reaction is carried out in the pH condition between 8.5 and 9.5 at 25° C. when mixed with the sample 2.

The pH buffer solution may be a combination of tris(hydroxymethyl)aminomethane and a mineral acid such as hydrochloric acid, nitric acid or sulfuric acid, as well as various pH buffer solutions. The buffer solution having adjusted pH is preferably used at a concentration between 10 mM and 100 mM in the PCR reagent. The primer refers to an oligonucleotide acting as a starting point for DNA synthesis by the PCR. The primer may be synthesized or isolated from biological sources.

The synthase is an enzyme for synthesis of DNA by primer addition, and includes chemically synthesized synthases. Suitable synthase includes, but is not limited to, E. coli DNA polymerase I, E. coli DNA polymerase Klenow fragment, T4 DNA polymerase, Taq DNA polymerase, T. litoralis DNA polymerase, Tth DNA polymerase, Pfu DNA polymerase, Hot Start Taq polymerase, KOD DNA polymerase, EX Taq DNA polymerase, and a reverse transcriptase. The term “thermostable” means the property of a compound which maintains its activity even at high temperatures, preferably between 65° C. and 95° C.

In the PCR step, the PCR is caused to occur in a mixture solution of the biological sample 2 and the PCR reagent 4 according to a predetermined temperature cycle. The PCR temperature cycle includes 3 steps, which are denaturation, primer adhesion (annealing) and primer extension, and this cycle is repeated whereby DNA is amplified. In one example of the steps, the denaturation step is carried out at 94° C. for 1 minute, the primer adhesion step at 55° C. for 1 minute, and the primer extension at 72° C. for 1 minute. The sample may be subjected to a genome extraction procedure; however, the one that is not subjected to the genome extraction procedure is used herein. Even with the biological sample not subjected to the genome extraction procedure, DNA is released from blood cells or cells at high temperature in the PCR temperature cycle, and the reagents necessary for the PCR come into contact with the DNA to make the reaction proceed.

After the PCR reaction is finished, an invader reagent 6 is added. A fluorescence-emitting FRET probe and cleavase (structure-specific DNA degradative enzyme) are contained in the invader reagent 6. The FRET probe is a fluorescent-labeled oligo having a sequence completely irrelevant to the genome DNA, and, irrespective of the type of SNP, its sequence is common.

Next, the reaction solution to which the invader reagent 6 has been added is reacted by addition to a plurality of probe arrangement parts 8. At each site of the probe arrangement parts 8, an invader probe and a reporter probe are individually held correspondingly to each of a plurality of SNP sites, and the reaction solution reacts with the invader probe to emit fluorescence if SNP corresponding to the reporter probe is present.

The invader method is described in detail in paragraphs [0032] to [0034] in Patent Literature 3.

Two reporter probes have been prepared depending on each base of SNP and can judge whether the SNP is a homozygote or heterozygote.

The invader method used in the typing step is a method of typing SNP site by hybridizing an allele-specific oligo with DNA containing SNP as an object of typing, wherein DNA containing SNP as an object of typing, two kinds of reporter probes specific to the each allele of SNP as an object of typing, one kind of invader probe, and an enzyme having a special endonuclease activity by which a structure of DNA is recognized and cleaved are used (see Patent Literature 3).

In the reaction vessel of the present invention, the film is preferably penetrable by a nozzle.

Among the reaction parts, at least one into which a nonvolatile liquid is to be dispensed is preferably formed into a concave portion capable of holding the nonvolatile liquid.

Also, a sample injection part into which a sample is injected may be formed as a concave portion in the same substrate.

It is preferred that at least the reaction part is covered with a detachable sealing material before use.

The typing reagent is an invader reagent or a TaqMan PCR reagent

In order to achieve the second object, the reaction vessel processing apparatus of the present invention has a reaction vessel mounting part provided for mounting a reaction vessel at least having a reaction part for allowing reaction of a sample, and a nonvolatile liquid reservoir part reserving a nonvolatile liquid having a lower specific gravity than the reaction solution; a dispenser 112 equipped with a mechanism for aspiration and discharge by a nozzle 28, as shown in FIG. 1, for conveying and dispensing a liquid; and a controller 118 for at least controlling a dispensing operation of the dispenser 112.

The reaction vessel processing apparatus may further have a reaction temperature control part for controlling temperature of the reaction part, and hence the controller 116 can also control temperature of the reaction temperature control part.

When the reaction vessel processing apparatus is used as a gene polymorphism detecting apparatus, the first aspect thereof uses a gene polymorphism diagnosing reaction vessel which further has a typing reagent reservoir part for reserving a typing reagent, and has a plurality of probe arrangement parts, each individually carrying a probe that emits fluorescence in correspondence with each of a plurality of polymorphic sites as the reaction part. As shown in FIG. 1, further provided as the reaction temperature control part, is a typing reaction temperature control part 110 that controls temperature of the probe arrangement part to such a temperature that allows a reaction solution of the sample and the typing reagent to react with probes, and the reaction vessel processing apparatus further has a fluorescence detector 64 for detecting fluorescence upon irradiation of each probe arrangement part with exciting light The controller 118 controls temperature of the typing reaction temperature control part 110 and a detection operation of the fluorescence detector 64.

When an invader reaction is used as the typing reaction, the typing reaction temperature control part 110 functions as a temperature regulation part for the invader reaction.

The second aspect that uses this reaction vessel processing apparatus as a gene polymorphism detecting apparatus uses a gene polymorphism diagnosing reaction vessel which further has a gene amplification reagent reservoir part for reserving a gene amplification reagent containing a plurality of primers to bind to a plurality of polymorphic sites by sandwiching each site between the primers, and has, as the reaction part, an amplification reaction part provided for allowing a gene amplification reaction for a mixture solution of the gene amplification reagent and the sample. As shown in FIG. 1, further provided as a reaction temperature control part, is an amplification reaction temperature control part 120 that controls temperature of the amplification reaction part to temperature for gene amplification for amplification of DNA in the reaction solution of the sample and the gene amplification reagent, and the controller 118 also controls temperature of the amplification reaction temperature control part 120.

When a PCR is used as a gene amplification reaction, the amplification reaction temperature control part 120 serves as a temperature regulation part for temperature cycle for the PCR.

For operating the controller 118 externally or displaying test results, a personal computer (PC) 122 may be connected to the controller 118.

In one exemplary nozzle, a disposable tip is removably attached to its tip end. When the liquid reservoir part of the reaction vessel is sealed with a film, and mounting to the reaction vessel processing apparatus is made in such a sealed condition with the film, aspiration of liquid is made through the film of the reaction vessel with the tip.

The diagnostic apparatus of the present invention for achieving the third object has a reaction vessel processing apparatus of the present invention for processing the gene polymorphism diagnosing reaction vessel among the reaction vessels of the present invention; a database storing diagnostic values for specific polymorphism or for combination of plural polymorphisms; a display; and a diagnosis processing apparatus that reads a diagnostic value from the database based on a polymorphism analysis result detected by the reaction vessel processing apparatus, and displays it on the display.

### EFFECTS OF THE INVENTION

Since the reaction vessel of the present invention holds a reaction part and a nonvolatile liquid having a lower specific gravity than the reaction solution in one substrate, by covering the surface of the reaction solution with the nonvolatile liquid in the reaction part, it is possible to prevent the reaction solution from evaporating even when the reaction solution is heated in the reaction part.

Further, provision of the reagent reservoir part realizes a reagent kit for reaction of a sample and eliminates the complexity in separately placing the reagent.

Since the first aspect using the reaction vessel as a gene polymorphism diagnosing reagent kit has a typing reagent reservoir part, a nonvolatile liquid reservoir part, and a probe arrangement part in an integrated manner, it is possible to conduct typing simultaneously for polymorphic sites for a DNA sample in which a plurality of polymorphic sites are amplified, and hence it is possible to achieve typing of a polymorphism in short time through a simple process.

Further, since the second aspect using the reaction vessel as a gene polymorphism diagnosing reagent kit, also has a gene amplification reagent reservoir part and an amplification reaction part in an integrated manner, it is possible to conduct simultaneous typing for a plurality of polymorphic sites after simultaneous amplification of the objective plural polymorphic sites from a biological sample, and hence it is possible to achieve typing of a polymorphism in short time through a simple process.

By making the film that seals the reagent or the nonvolatile liquid penetrable by a nozzle, transfer of a liquid in the reaction vessel processing apparatus is facilitated.

By making the reaction part as a concave portion capable of holding the nonvolatile liquid, it is possible to prevent the reaction solution from evaporating in the reaction part more effectively.

When the reaction part is covered with a detachable sealing material, it is possible to prevent adhesion of dirt or stain before use by covering the reaction part with the sealing material before use and removing the sealing material at the time of use.

In the reaction vessel having an amplification reaction part, by making the liquid dispensing port of the amplification reaction part have an opening shape corresponding to the shape of the tip end of the dispensing nozzle and making it with an elastic material capable of closely fitting with the tip end of the dispensing nozzle, it is possible to facilitate a dispensing operation of the mixture solution to the amplification reaction part and collection of the reaction solution from the amplification reaction part.

In the reaction vessel processing apparatus of the present invention, since a liquid is transferred by a nozzle, it is possible to realize a dispensing operation with a simple mechanism.

In the diagnostic apparatus of the present invention, it is possible to automatically execute the process from polymorphism typing to display of diagnostic values based on the same.

### BEST MODE FOR CARRYING OUT THE INVENTION

As a nonvolatile liquid having a lower specific gravity than a reaction solution, mineral oil, vegetable oil, animal oil, silicone oil, or diphenylether may be used. Mineral oil is a liquid hydrocarbon mixture obtained by distillation from petrolatum, and is also called liquid paraffin, liquid petrolatum, white oil and the like, and includes light oil of low specific gravity. Examples of animal oil include cod-liver oil, halibut oil, herring oil, orange roughy oil, shark liver oil, and the like. Examples of vegetable oil include canola oil, almond oil, cotton seed oil, corn oil, olive oil, peanut oil, safflower oil, sesame oil, soybean oil, and the like.

FIG. 2A and FIG. 2B show the first example of the reaction vessel, wherein FIG. 2A is a front view, and FIG. 2B is a plan view.

On the same side of a plate-like substrate 10, a reagent reservoir part 14 and a nonvolatile liquid reservoir part 16 are formed as concave portions. As the nonvolatile liquid, mineral oil is used, and hereinafter, the nonvolatile liquid reservoir part is referred to as a mineral oil reservoir part. On the same side of the substrate 10, further formed is a reaction part 18. The reagent reservoir part 14 and the mineral oil reservoir part 16 are sealed with a film 20, and for aspirating the reagent and the mineral oil and transferring them to other locations by a nozzle, they are aspirated by a nozzle after removal of the film 20, or the film 20 that is adapted to be penetrable by a nozzle is penetrated by the nozzle and the reagent and the oil are aspirated by the nozzle. Such film 20 may be implemented, for example, by an aluminum foil or a laminate film of a resin film such as a PET (polyethylene terephthalate) film, and bonded by fusion or adhesion so that it will not detach easily.

The surface of the substrate 10 is covered from above the film 20 with a detachable sealing material 22 of the size that covers the reagent reservoir part 14, the mineral oil reservoir part 16, and the reaction part 18.

One example of concrete use of the reaction vessel is a gene polymorphism diagnosing reagent kit in which a sample reaction solution having DNA amplified by a PCR is dispensed and SNP is detected by an invader reaction. Referring to FIG. 2A and FIG. 2B, an example as the gene polymorphism diagnosing reagent kit will be explained in detail.

On the same side of the plate-like substrate 10, a sample injection part 12, the typing reagent reservoir part 14, and the mineral oil reservoir part 16 are formed as concave portions. On the same side of the substrate 10, further formed is a plurality of probe arrangement parts 18.

A biological sample reaction solution having DNA amplified by a PCR will be injected to the sample injection part 12; however in the condition before use, the sample injection part 12 is provided in an empty state in which a sample is not injected. The typing reagent reservoir part 14 reserves 10 μL to 300 μL of a typing reagent that is prepared in correspondence with a plurality of polymorphic sites, and the mineral oil reservoir part 16 reserves 20 μL to 300 μL of mineral oil for preventing evaporation of the reaction solution. The typing reagent reservoir part 14 and mineral oil reservoir part 16 are sealed with the film 20 which is penetrable by a nozzle.

Each probe arrangement part 18 individually has a probe that emits fluorescence in correspondence with each of plural polymorphic sites, and is a concave portion capable of holding the mineral oil when it is dispensed from the mineral oil reservoir part 16. Each concave portion of the probe arrangement part 18 is, for example, in the shape of a circle of 100 μm to 2 mm in diameter, and 50 μm to 1.5 mm in depth.

The surface of the substrate 10 is covered from above the film 20 with the detachable sealing material 22 of the size that covers the sample injection part 12, the typing reagent reservoir part 14, the mineral oil reservoir part 16 and the probe arrangement part 18. This sealing material 22 may also be an aluminum foil or a laminate film of aluminum and a resin; however, the bonding strength is smaller than that of the film 20 and is bonded by an adhesive or the like in such a degree that it can be detached.

In order to measure fluorescence from the bottom face side, the substrate 10 is made of a light-permeable resin with a low-spontaneous-fluorescent property (that is, a property of generating little fluorescence from itself), for example, a material such as polycarbonate. The thickness of the substrate 10 is 0.3 mm to 4 mm, and preferably 1 mm to 2 mm. From the viewpoint of the low-spontaneous-fluorescent property, it is preferred that the thickness of the substrate 10 is as small as possible.

A method of using the reaction vessel according to the present example will be described.

As shown in FIG. 3, the sealing material 22 is detached at the time of use. The film 20 that seals the typing reagent reservoir part 14 and the mineral oil reservoir part 16 is not detached and still remains.

To the sample injection part 12, 2 μL to 20 μL of a sample reaction solution 24 having DNA amplified externally by a PCR reaction is injected with a pipette 26 or the like. Then the reaction vessel is mounted on the detecting apparatus.

In the detecting apparatus, as shown in FIG. 4, a typing reagent is aspirated by the nozzle 28 inserted into the typing reagent reservoir part 14 through the film 20, and the typing reagent is transferred to the sample injection part 12 by the nozzle 28. In the sample injection part 12, the sample reaction solution and the typing reagent are mixed by repetition of aspiration and discharge by the nozzle 28.

Thereafter, the reaction solution of the PCR solution and the typing reagent is dispensed to each probe arrangement part 18 by the nozzle 28. To each probe arrangement part 18, mineral oil is dispensed from the mineral oil reservoir part 16 by the nozzle 28. Dispensing of mineral oil to the probe arrangement part 18 may be conducted before dispensing of the reaction solution to the probe arrangement part 18. To each probe arrangement part 18, 0.5 μL to 10 μL of mineral oil is dispensed, and the mineral oil covers the surface of the reaction solution. As a result, it is possible to prevent the reaction solution from evaporating during typing reaction time which is associated with heat generation at the typing reaction temperature control part of the detecting apparatus.

In each probe arrangement part 18, the reaction solution and the probe react, and if a predetermined SNP is present, fluorescence is emitted from the probe. Fluorescence is detected upon irradiation with exciting light from the back face side of the substrate 10.

FIG. 5A, FIG. 5B and FIG. 5C show a second example of the reaction vessel.

FIG. 5A is a front view, FIG. 5B is a plan view, and FIG. 5C is an enlarged section view along the line X-X in FIG. 5B.

In this reaction vessel, a biological sample not subjected to a nucleic acid extraction procedure is injected as a sample, and both amplification of DNA by a PCR reaction and SNP detection by an invader reaction are conducted. It is to be noted, however, a biological sample not subjected to a nucleic acid extraction procedure may be injected.

On the same side of a plate-like substrate 10a, the sample injection part 12, the typing reagent reservoir part 14, the mineral oil reservoir part 16, and the plurality of probe arrangement parts 18 similar to those in the example of FIG. 2A and FIG. 2B are formed. In this reaction vessel, on the same side of the substrate 10a, a gene amplification reagent reservoir part 30, a PCR-finished solution injection part 31, and an amplification reaction part 32 are also formed.

The gene amplification reagent reservoir part 30 is also formed as a concave portion in the substrate 10a, and reserves a gene amplification reagent containing a plurality of primers to bind to a plurality of polymorphic sites by sandwiching each site between the primers. The gene amplification reagent reservoir part 30, the typing reagent reservoir part 14 and the mineral oil reservoir part 16 are sealed with the film 20 which is penetrable by a nozzle. The gene amplification reagent reservoir part 30 reserves 2 μL to 300 μL of a PCR reagent. In the same way as the example shown in FIG. 2A and FIG. 2B, the typing reagent reservoir part 14 reserves 10 μL to 300 μL of a typing reagent, and the mineral oil reservoir part 16 reserves 20 μL to 300 μL of mineral oil.

The PCR-finished solution injection part 31 is provided for mixing the reaction solution having finished a PCR reaction in the amplification reaction part 32 and the typing reagent, and is formed as a concave portion in the substrate 10a, and provided in an empty state before use.

The amplification reaction part 32 allows the mixture solution of the PCR reagent and the sample to proceed a gene amplification reaction.

FIG. 6 is an enlarged section view of a part of the amplification reaction part 32. FIG. 6 is a section view along the line Y-Y in FIG. 5B. As shown in FIG. 6, liquid dispensing ports 34a, 34b of the amplification reaction part 32 have openings 36a, 36b having the shape corresponding to the shape of a tip end of the nozzle 28, and are made of an elastic material such as PDMS (polydimethylsiloxane) or silicone rubber for allowing close fitting to the tip end of the nozzle 28.

The amplification reaction part 32 has a smaller thickness in the bottom face side of the substrate 10a so as to improve the heat conductivity, as shown in FIG. 5C and FIG. 6. The thickness of that part is, for example, 0.2 mm to 0.3 mm.

To the sample injection part 12, a biological sample not subjected to a nucleic acid extraction procedure is injected in the present example; however, it is provided in an empty state where a sample is not injected before use.

In the same way as the example shown in FIG. 2A and FIG. 2B, the typing reagent reservoir part 14 reserves a typing reagent that is prepared in correspondence with a plurality of polymorphic sites, and the mineral oil reservoir part 16 reserves mineral oil for preventing vaporization of the reaction solution.

In the same way as the example shown in FIG. 2A and FIG. 2B, each probe arrangement part 18 individually holds a probe that emits fluorescence in correspondence with each of the plurality of polymorphic sites, and is formed as a concave portion capable of holding mineral oil when the mineral oil is dispensed from the mineral oil reservoir part 16.

The surface of the substrate 10a is covered from above the film 20, with the sealing material 22 which can be detached and has such a size that covers the sample injection part 12, the PCR-finished solution injection part 31, the typing reagent reservoir part 14, the mineral oil reservoir part 16, the gene amplification reagent reservoir part 30, the amplification reaction part 32 and the probe arrangement part 18. The materials and the manner of bonding the film 20 and the sealing material 22 are as described in the example of FIG. 2A and FIG. 2B.

In order to also measure fluorescence from the bottom side, the substrate 10a is made of a light-permeable resin with a low-spontaneous-fluorescent property, for example, a material such as polycarbonate. The thickness of the substrate 10 is 1 to 2 mm.

The manner of using the reaction vessel according to the present example is shown below.

As shown in FIG. 7A and FIG. 7B, the sealing material 22 is detached at the time of use. The film 20 that seals the typing reagent reservoir part 14, the mineral oil reservoir part 16, and the gene amplification reagent reservoir part 30 is not detached and still remains.

To the sample injection part 12, 0.5 μL to 2 μL of a sample 25 is injected with a pipette 26 or the like. In the example of FIG. 2A and FIG. 2B, the injected sample is a sample reaction solution having DNA amplified externally by a PCR reaction; however, the sample injected in the present example is a biological sample, for example, blood, not subjected to a nucleic acid extraction procedure. The sample may be a biological sample subjected to a nucleic acid extraction procedure. After application of the sample, the reaction vessel is mounted on a detecting apparatus.

In the detecting apparatus, as shown in FIG. 8A and FIG. 8B, the nozzle 28 is inserted into the gene amplification reagent reservoir part 30 through the film 20 and the PCR reagent is aspirated, and 5 μL to 20 μL of the PCR reagent is transferred to the sample injection part 12 by the nozzle 28. In the sample injection part 12, the sample reaction solution and the PCR reagent are mixed to form a PCR solution by repetition of aspiration and discharge by the nozzle 28.

Next, as shown in FIG. 6A, the PCR solution is injected to the amplification reaction part 32 by the nozzle 28. That is, the nozzle 28 is inserted into one port 34a of the amplification reaction part 32 and the PCR solution 38 is injected, and then mineral oil 40 is injected to the ports 34a, 34b by the nozzle 28 so as to prevent the PCR solution 38 from evaporating during reaction in the amplification reaction part 32, whereby surfaces of the PCR solution 38 in the ports 34a, 34b are covered with the mineral oil 40.

After completion of the PCR reaction, the PCR solution is collected by the nozzle 28, and at this time, mineral oil 40 is injected through one port 34a of the amplification reaction part 32 as shown in FIG. 6B so as to facilitate the collection. A reaction-finished PCR solution 38a is pushed to the other port 34b. Then the nozzle 28 is inserted and the PCR solution 38a is aspirated into the nozzle 28. Since the ports 34a, 34b have openings 36a, 36b that are formed in correspondence with the shape of the nozzle 28, and made of an elastic material, the nozzle 28 comes into close contact with the ports 34a, 34b to prevent liquid leakage, and facilitate an operation of application and collection of the PCR solution.

The reaction-finished PCR solution 38a collected from the amplification reaction part 32 by the nozzle 28 is transferred and injected to the PCR-finished solution injection part 31.

Next the nozzle 28 is inserted into the typing reagent reservoir part 14 through the film 20 and the typing reagent is aspirated, and the typing reagent is transferred and injected to the PCR-finished solution injection part 31 by the nozzle 28. In the PCR-finished solution injection part 31, the PCR solution and the typing reagent are mixed by repetition of aspiration and discharge by the nozzle 28.

Then, 0.5 μL to 4 μL of the reaction solution of the PCR solution and the typing reagent is dispensed to each probe arrangement part 18 by the nozzle 28. To each probe arrangement part 18, mineral oil is dispensed by the nozzle 28 from the mineral oil reservoir part 16. Dispensing of mineral oil to the probe arrangement part 18 may be conducted before dispensing of the reaction solution to the probe arrangement part 18. In each probe arrangement part 18, the mineral oil covers the surface of the reaction solution, to prevent the reaction solution from evaporating during the period of typing reaction by the typing reaction temperature control part of the detecting apparatus, which is associated with heat generation.

In each probe arrangement part 18, the reaction solution and the probe react, and if a predetermined SNP is present, fluorescence is emitted from the probe. Fluorescence is detected upon irradiation with exciting light from the back-face side of the substrate 10.

In the following, the present invention will be described in detail while showing a composition of each reaction reagent; however, the technical scope of the present invention is not limited by these examples.

The PCR reagent is known in the art, and a reaction reagent containing a primer, DNA polymerase and TaqStart (available from CLONTECH Laboratories) as described in Patent document 3, paragraph [0046], for example, may be used. Further, AmpDirect (available from SHIMADZU Corporation) may be contained in the PCR reagent As the primer, for example, SNP IDs 1 to 20, SEQ No. 1 to 40 described in Table 1 in Patent document 3 may be used.

As the typing reagent, an invader reagent is used. As the invader reagent, an invaderassay kit (available from Third Wave Technology) is used. For example, a signal buffer, an FRET probe, a structure specific DNase and an allele specific probe are prepared in concentrations as described in Patent document 3, paragraph [0046].

FIG. 9 shows one example of a simplified reaction vessel processing apparatus that uses the reaction vessel of the present invention as a reagent kit and detects SNP of a biological sample. In the apparatus, a pair of upper and lower heat blocks 60 and 62 is disposed to constitute a mounting part for a reaction vessel, and five reaction vessels 41 of the present invention into which a sample is injected are arranged in parallel on the lower heat block 60. These heat blocks 60, 62 are able to move in the Y direction represented by the arrow.

The upper heat block 62 is formed with an openable and closable window so that a lid can be open at the time of transfer, aspiration, or discharge of a liquid by the nozzle 28.

The lower heat block 60 has an amplification reaction temperature control part that controls temperature of the amplification reaction part 32 to achieve a predetermined temperature cycle, and a typing reaction temperature control part that controls temperature of the probe arrangement part 18 to a temperature that causes a reaction between DNA and a probe. The temperature of the amplification reaction temperature control part is set so that it is varied at three stages, for example, 94° C., 55° C. and 72° C. in this order, and the cycle is repeated. The temperature of the typing reaction temperature control part is set, for example, at 63° C.

When the reaction vessel 41 not having an amplification reaction part as is the case of the example shown in FIG. 2A and FIG. 2B is used, the amplification reaction temperature control part for controlling temperature of the amplification reaction part is not needed.

Below the heat block 60, a detector 64 for detecting fluorescence is disposed, and the detector 64 moves in the direction of the arrow X in the figure and detects fluorescence from the probe arrangement part 18. The heat block 60 is provided with an opening for detection of fluorescence. Fluorescence detection in each probe is achieved by a Y-directional movement of the probe arrangement part 18 by the reaction vessel mounting part and an X-directional movement of the detector 64.

For achieving transfer, aspiration, or discharge of a liquid by the nozzle 28, a liquid feeding arm 66 is provided as a dispenser, and the liquid feeding arm 66 has a nozzle 28. To a tip end of the nozzle 28, a disposable tip 70 is detachably mounted.

In order to control operations of the heat blocks 60, 62, the fluorescence detector 64 and the liquid feeding arm 66, a controller 118 is disposed near these elements. The controller 118 has a CPU and stores a program for operation. The controller 118 controls temperature control of the typing reaction part 110 and the amplification part 120, which are realized by the heat blocks 60, 62, a detection operation of the fluorescence detector 64, and a dispensing operation of the liquid feeding arm 66 of the dispenser 112.

When the reaction vessel 41 not having a gene amplification reaction part as in the case of the reaction vessel of FIG. 2A and FIG. 2B is used, the amplification part that controls temperature of the gene amplification reaction part is not needed, and there is no need for the controller 118 to have the function for temperature control of the amplification part.

FIG. 10 shows the details of the detector 64. The detector 64 includes a laser diode (LD) or light-emitting diode (LED) 92 as an exciting light source for emitting a laser light at 473 nm, and a pair of lenses 94, 96 for applying the laser light after collecting it on the bottom face of the probe arrangement part of the reaction vessel 41. The lens 94 is a lens for collecting the laser light from the laser diode 92 to convert it into a parallel light. The lens 96 is an objective lens for applying the parallel light after converging it on the bottom face of the reaction vessel 41. The objective lens 96 also functions as a lens for collecting fluorescence emitted from the reaction vessel 41. Between the pair of lenses 94, 96, a dichroic mirror 98 is provided, and wavelength characteristics of the dichroic mirror 98 is established so that an exciting light passes therethrough, while fluorescent light is reflected. On the optical path of a reflected light (fluorescence) of the dichroic mirror 98, a further dichroic mirror 100 is disposed. Wavelength characteristics of the dichroic mirror 100 are established so that a light at 525 nm is reflected, while a light at 605 nm passes therethrough. On the optical path of a light reflected by the dichroic mirror 100, a lens 102, and an optical detector 104 are arranged so as to detect fluorescent light of 525 nm, and on the optical path of a light transmitted the dichroic mirror 100, a lens 106 and an optical detector 108 are arranged so as to detect fluorescent light at 605 nm. By detecting two kinds of fluorescence with the two detectors 104, 108, the presence or absence of SNP corresponding to the invader probe fixed in each probe array position, and whether the SNP is a homozygote or a heterozygote are detected. As a labeled fluorescent substance, for example, FAM, ROX, VIC, TAMRA, Redmond Red and the like may be used.

The detector 64 of FIG. 10 is designed to measure fluorescence of two wavelengths upon irradiation with an exciting light from a single light source; however, the detector 64 may also be designed to use two light sources for enabling irradiation with different exciting wavelengths for fluorescence measurement at two wavelengths.

As shown in FIG. 12, the diagnostic apparatus of the present invention is made up of a reaction vessel processing apparatus 200 for processing the gene polymorphism diagnosing reaction vessel among the reaction vessels of the present invention, a database 202 consisting of a storage apparatus such as a disc apparatus or a drum device, having storing diagnostic values about a specific polymorphism or a combination of plural polymorphisms, a display 204 such as a liquid crystal display or a CRT, and a diagnosis processing apparatus 206 consisting of a computer that reads out diagnostic values from the database 202 based on a result of polymorphism analysis detected by the reaction vessel processing apparatus 200 and displays the value on the display 204.

### INDUSTRIAL APPLICABILITY

The present invention may be utilized in various types of automatic analyses, for example, in research of gene analysis or clinical field, as well as in measurement of various chemical reactions. For example, the present invention can be used in detecting genome DNA polymorphism for plants and animals including humans, particularly SNP and can further be utilized, not only in diagnosing disease morbidity, the relationship between the type and effect or side effect of a drug administered and so on by using the results of the above detection, but also in judgment of the variety of animal, or plant, diagnosis of injections (judgment of the type of invader) etc.

### BRIEF DESCRIPTION OF THE DRAWINGS

FIG. 1 A block diagram schematically showing the present invention.

FIG. 2A A front view of the first example of a reaction vessel.

FIG. 2B A plan view of the first example of the reaction vessel.

FIG. 3A A front view showing a former half of a process of an SNP detection method using the reaction vessel of the same example.

FIG. 3B A plan view showing the former half of the process of the SNP detection method using the reaction vessel of the same example.

FIG. 4A A front view showing a latter half of the process of the SNP detection method using the reaction vessel of the same example.

FIG. 4B. A plan view showing the latter half of the process of the SNP detection method using the reaction vessel of the same example.

FIG. 5A A front view showing the second example of the reaction vessel.

FIG. 5B A plan view showing the second example of the reaction vessel.

FIG. 5C An enlarged section view along the line X-X in FIG. 5B showing the second example of the reaction vessel.

FIG. 6A An enlarged section view of an amplification reaction part in the same example along the line Y-Y of FIG. 5B in the condition that a reaction solution is injected.

FIG. 6B An enlarged section view of the amplification reaction part in the same example along the line Y-Y of FIG. 5B in the condition that the reaction solution is collected.

FIG. 7A A front view showing a former half of the process of the SNP detection method using the reaction vessel of the same example.

FIG. 7B A plan view showing the former half of the process of the SNP detection method using the reaction vessel of the same example.

FIG. 8A A front view showing a latter half of the process of the SNP detection method using the reaction vessel of the same example.

FIG. 8B A plan view showing the latter half of the process of the SNP detection method using the reaction vessel of the same example.

FIG. 9 A schematic perspective view showing one example of a simplified reaction vessel processing apparatus that uses the reaction vessel of the present invention as a reagent kit, and detects SNP of a biological sample.

FIG. 10 A schematic structure view showing a detector in the same detecting apparatus.

FIG. 11 A flow chart schematically showing an SNP detection method which may be related to the present invention.

FIG. 12 A block diagram schematically showing a diagnostic apparatus of the present invention.

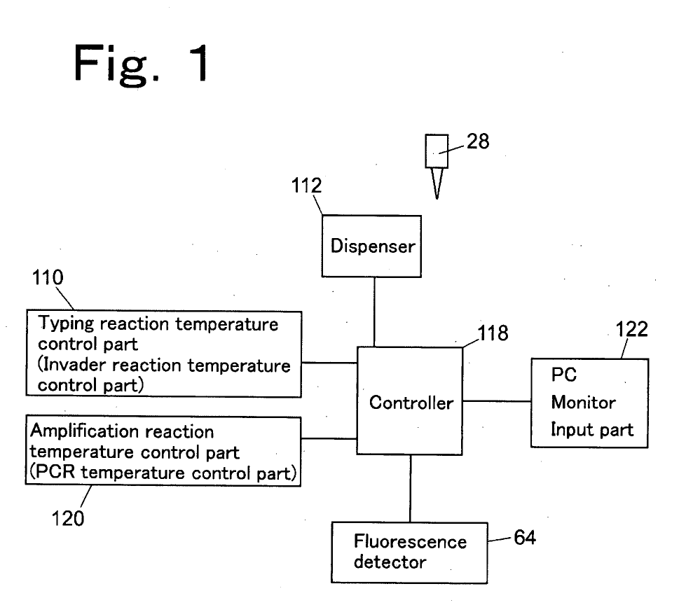

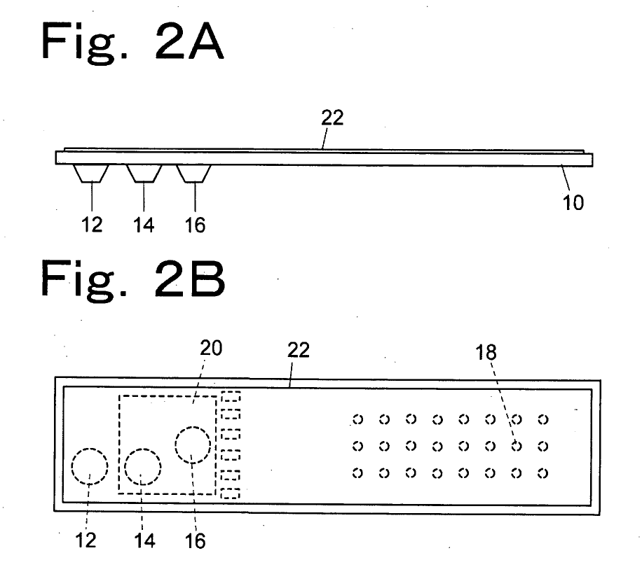

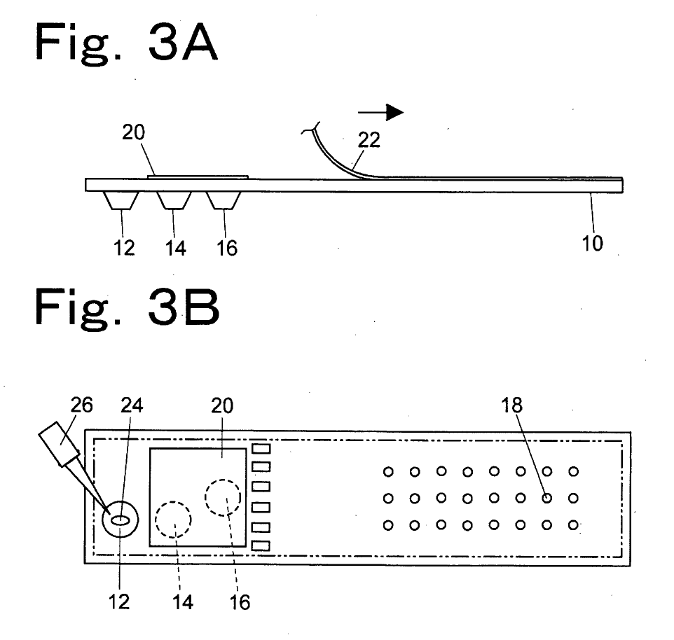

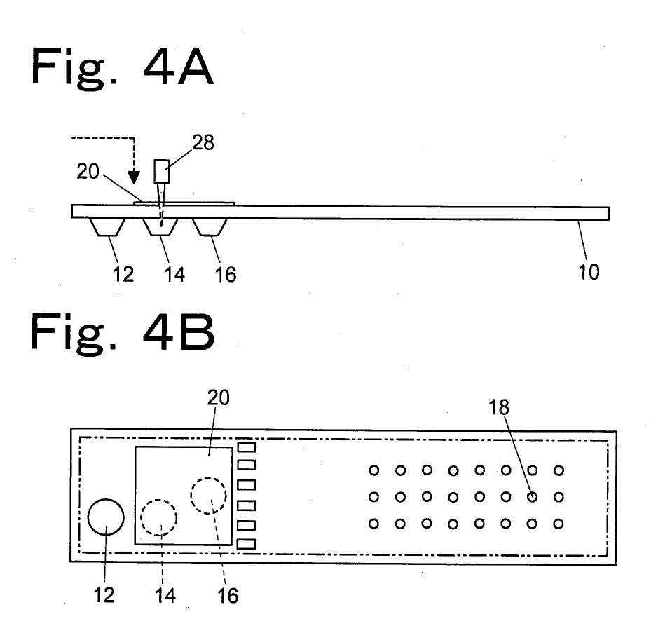

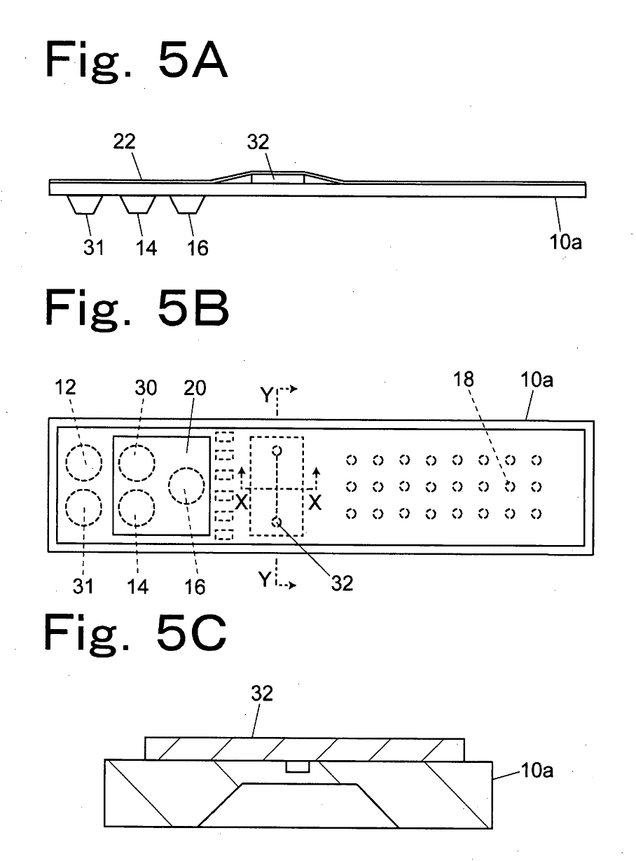

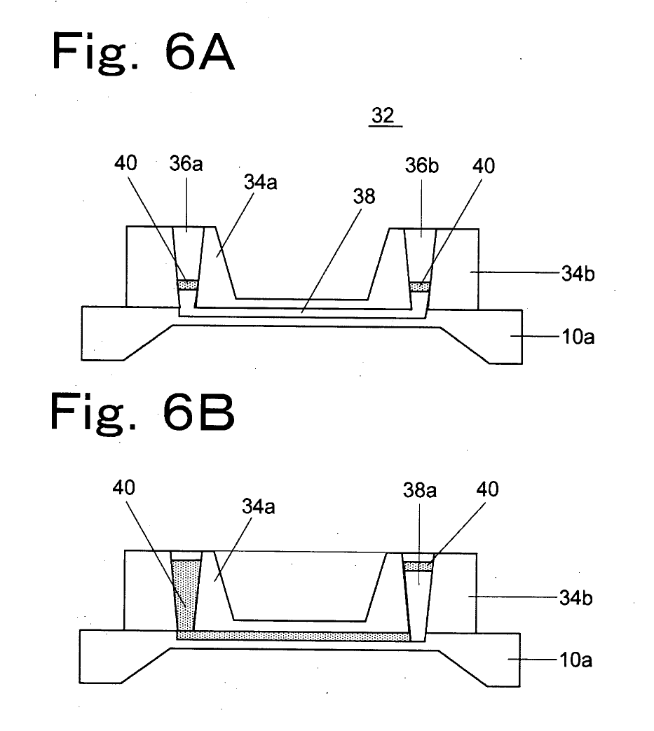

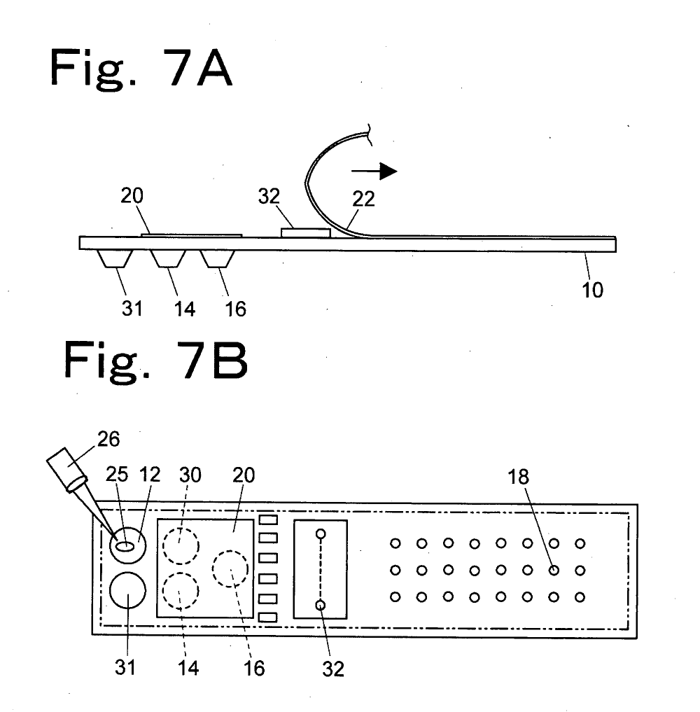

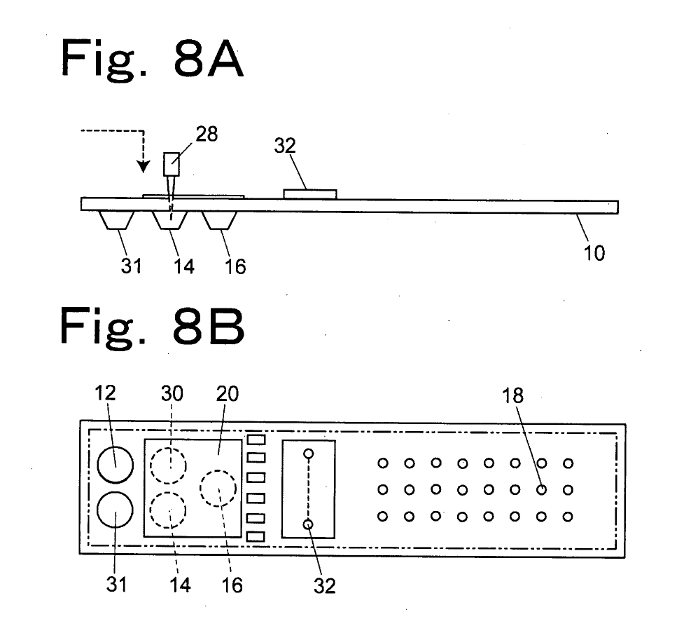

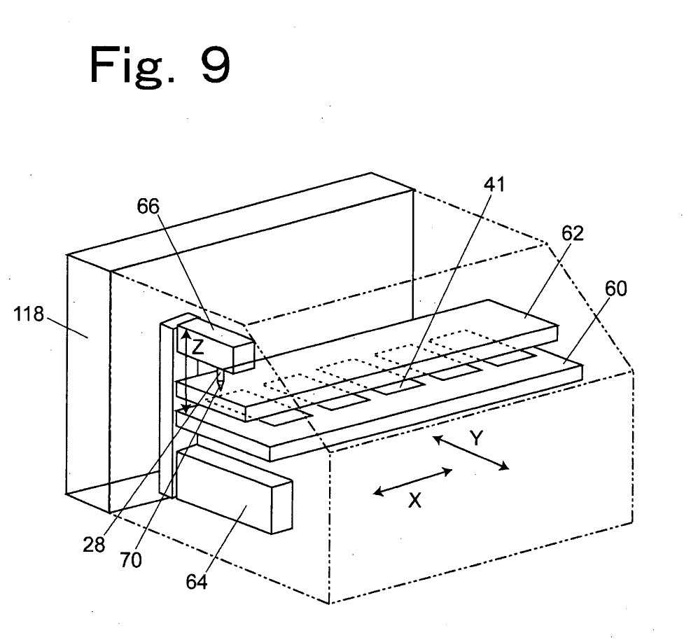

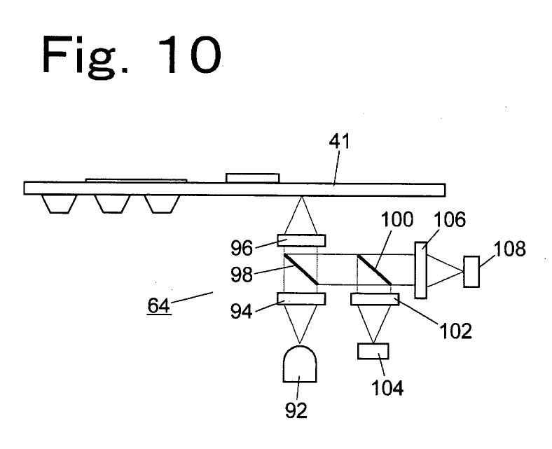

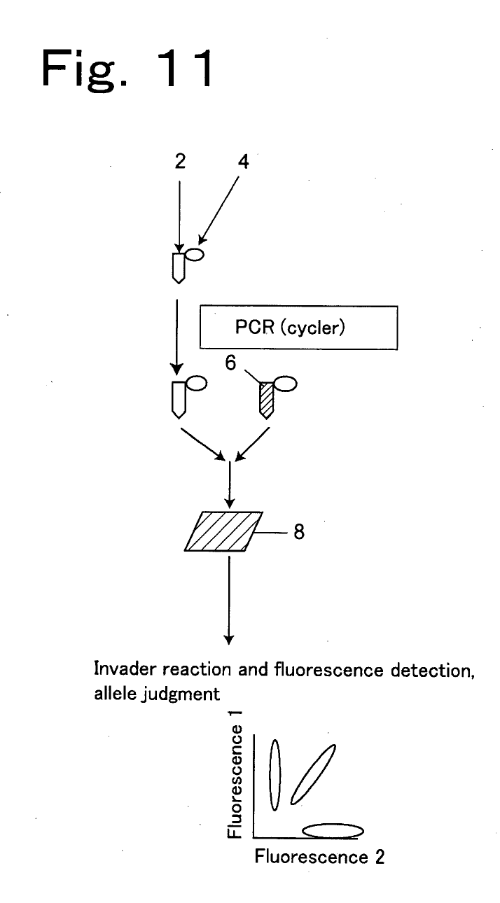

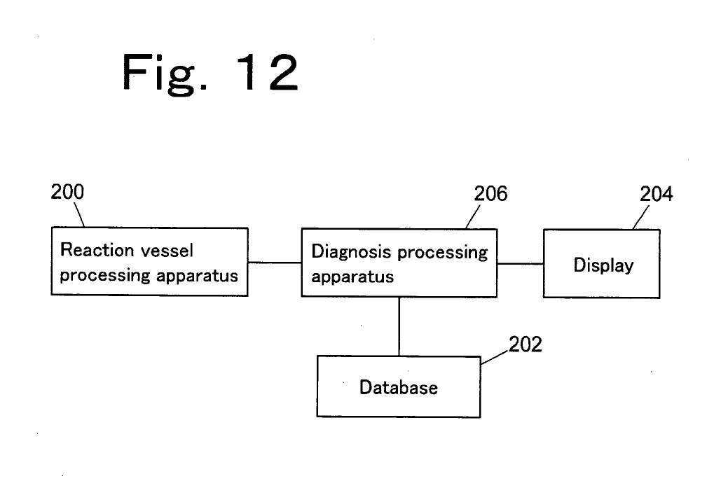

### DESCRIPTION OF THE REFERENCE NUMERALS

2 sample
4 PCR reagent
6 invader reagent
8 probe arrangement part
10,10a substrate
12 sample injection part
14 typing reagent reservoir part
16 mineral oil reservoir part
18 probe arrangement part
20 film
22 sealing material
28 nozzle
30 gene amplification reagent reservoir part
31 PCR-finished solution injection part
32 amplification reaction part
34a, 34b port of amplification reaction part
36a, 36b opening of port
41 reaction vessel
60, 62 heat block
64 detector
66 liquid feeding arm
70 tip
200 reaction vessel processing apparatus
202 database
204 display
206 diagnosis processing apparatus

1. A reaction vessel comprising:
at least one reaction part formed on a plate-like substrate, for allowing a reaction of a sample, and
a nonvolatile liquid reservoir part formed as a concave portion in the substrate, which holds a nonvolatile liquid having a lower specific gravity than a reaction solution, and is sealed with a film.

2. The reaction vessel according to claim 1, further comprising at least one reagent reservoir part which is formed as a concave portion in the substrate, reserves a reagent used for a reaction of the sample, and is sealed with a film, whereby a regent kit for reaction of a sample is formed.

3. The reaction vessel according to claim 2, further comprising:
as the reagent reservoir part, a typing reagent reservoir part for reserving a typing reagent prepared in correspondence with a plurality of polymorphic sites, and
as the reaction part, a plurality of probe arrangement parts each individually holding a probe that emits fluorescence in correspondence with each of the plurality of polymorphic sites, whereby a gene polymorphism diagnosing reagent kit is formed.

4. The reaction vessel according to claim 3, further comprising:
as the reagent reservoir part, a gene amplification reagent reservoir part for reserving a gene amplification reagent containing a plurality of primers to bind to a plurality of polymorphic sites by sandwiching each site between the primers, and
as the reaction part, an amplification reaction part that allows a gene amplification reaction for a mixture solution of the gene amplification reagent and the sample.

5. The reaction vessel according to claim 4, wherein a liquid dispensing port of the amplification reaction part has an opening shape corresponding to the shape of a tip end of a dispensing nozzle, and made of an elastic material capable of closely fitting with the tip end of the dispensing nozzle.

6. The reaction vessel according to claim 4, wherein a thickness of the substrate in the amplification reaction part is smaller than a thickness of the remaining part of the substrate.

7. The reaction vessel according to claim 1, wherein the film is penetrable by a nozzle.

8. The reaction vessel according to claim 1, wherein of the reaction parts, at least one to which the nonvolatile liquid is dispensed is in the shape of a concave portion capable of holding the nonvolatile liquid.

9. The reaction vessel according to claim 1, further comprising a sample injection part formed as a concave portion in the substrate, to which a sample is to be injected.

10. The reaction vessel according to claim 1, wherein at least the reaction part is covered with a detachable sealing material before use.

11. The reaction vessel according to claim 1, wherein the nonvolatile liquid is a liquid selected from the group consisting of mineral oil, vegetable oil, animal oil, silicone oil and diphenylether.

12. The reaction vessel according to claim 3, wherein an objective polymorphism is a single-nucleotide polymorphism.

13. The reaction vessel according to claim 8, wherein the sample is a biological sample not subjected to a nucleic acid extraction procedure.

14. The reaction vessel according to claim 8, wherein the gene amplification reagent is a PCR reagent.

15. The reaction vessel according to claim 3, wherein the typing reagent is an invader reagent or a TaqMan PCR reagent.

16. A reaction vessel processing apparatus comprising:
a reaction vessel mounting part, for mounting a reaction vessel including at least a reaction part for allowing a reaction of a sample, and a nonvolatile liquid reservoir part which reserves a nonvolatile liquid having a lower specific gravity than a reaction solution,
a dispenser equipped with a mechanism for aspiration and discharge by a nozzle, for transferring and dispensing a liquid, and
a controller that controls at least a dispensing operation of the dispenser.

17. The reaction vessel processing apparatus according to claim 16, further comprising a reaction temperature control part that controls a temperature of the reaction part, wherein the controller also controls a temperature of the reaction temperature control part.

18. The reaction vessel processing apparatus according to claim 17, wherein the reaction vessel is a gene polymorphism diagnosing reaction vessel further including a typing reagent reservoir part for reserving a typing reagent, and as the reaction part, a plurality of probe arrangement parts each individually holding a probe that emits fluorescence in correspondence with each of a plurality of polymorphic sites,
the reaction vessel processing apparatus further comprising, as the reaction temperature control part, a typing reaction temperature control part that controls a temperature of the probe arrangement parts to such a temperature that causes a reaction solution of the sample and the typing reagent to react with the probe,
the reaction vessel processing apparatus further comprising a fluorescence detector for detecting fluorescence upon irradiation of each probe arrangement part with exciting light,
the controller controlling temperature control of the typing reaction temperature control part and a detection operation of the fluorescence detector.

19. The reaction vessel processing apparatus according to claim 18, wherein the reaction vessel is a gene polymorphism diagnosing reaction vessel further including a gene amplification reagent reservoir part that reserves a gene amplification reagent containing a plurality of primers to bind to a plurality of polymorphic sites by sandwiching each site between the primers, and as the reaction part, an amplification reaction part that allows a gene amplification reaction for a mixture solution of the gene amplification reagent and the sample,
reaction vessel processing apparatus further comprising, as the reaction temperature control part, an amplification reaction temperature control part that controls a temperature of the amplification reaction part to a temperature for gene amplification for amplifying DNA in a reaction solution of the sample and the gene amplification reagent,
the controller further controlling a temperature of the amplification reaction temperature control part.

20. The reaction vessel processing apparatus according to claim 16, wherein a disposable tip is detachably attached to a tip end of the nozzle, and the liquid reservoir part of the reaction vessel is sealed with a film, and the reaction vessel is mounted on the reaction vessel processing apparatus while it is sealed with the film, and a liquid is aspirated by the tip through the film.

21. A diagnostic apparatus comprising:
the reaction vessel processing apparatus according to claim 18;
a database storing diagnostic values for a specific polymorphism or for a combination of plural polymorphisms;
a display; and
a diagnosis processing apparatus that reads a diagnostic value from the database based on a polymorphism analysis result detected by the reaction vessel processing apparatus, and displays it on the display.
